(19) United States
(12) Patent Application Publication
(10) Pub. No.: US 2024/0057852 A1
(43) Pub. Date: Feb. 22, 2024

(54) TUBE CONNECTOR FOR ENDOSCOPE SYSTEMS

(71) Applicant: Boston Scientific Scimed, Inc., Maple Grove, MN (US)

(72) Inventors: Ryan V. Wales, Northborough, MA (US); Ryan Vincent William Pollock, Leominster, MA (US); John B. Golden, Norton, MA (US); Jeff Gray, Sudbury, MA (US); Scott E. Brechbiel, Acton, MA (US); Colby Harris, Norfolk, MA (US)

(73) Assignee: Boston Scientific Scimed, Inc., Maple Grove, MN (US)

(21) Appl. No.: 18/451,664

(22) Filed: Aug. 17, 2023

Related U.S. Application Data
(60) Provisional application No. 63/399,528, filed on Aug. 19, 2022.

(51) Int. Cl. A61B 1/00; A61B 1/12

(52) U.S. Cl. CPC A61B 1/00128; A61B 1/126

(57) ABSTRACT

Devices, systems, and methods for connecting tubes to an endoscope. A connector for connecting tubing, such as gas/lens wash tubing, to fluid ports of an endoscope may include a first end that couples with the tubing and a second end for engaging fluid ports of an endoscope. The connector may have a stabilizing component configured to facilitate securing the second end to the endoscope. In some cases, the connector may include a device coupling member configured to couple to the endoscope and a core structure adding stability to the device coupling member and configured to couple to the tubing. In some cases, the stabilizing component may include an actuator and a retention component that adjusts in response to actuation of the actuator.

### CROSS-REFERENCE TO RELATED APPLICATIONS

This application claims the benefit of U.S. Provisional Patent Application Ser. No. 63/399,528 filed on Aug. 19, 2022, the disclosure of which is incorporated herein by reference.

### TECHNICAL FIELD

This disclosure relates generally to connector assemblies and methods, and particularly to tubing connectors and methods for an endoscope system.

### BACKGROUND

A wide variety of intracorporeal and extracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a connector for connecting a gas/lens wash tubing to fluid lines of an endoscope may comprise a core configured to couple to the gas/lens wash tubing, a device coupling member coupled to the core, the device coupling member defining a first port configured to couple with a first fluid port of an endoscope and a second port configured to couple with a second fluid port of an endoscope, and a stabilizing member configured to facilitate securing the device coupling member to the endoscope.

Alternatively or additionally to any of the examples above, the stabilizing member may comprise a ring in the first port.

Alternatively or additionally to any of the examples above, the ring may have an inner surface and the inner surface tapers toward a central axis of the first port.

Alternatively or additionally to any of the examples above, the stabilizing member may include a ring and extends distally from the core into the first port.

Alternatively or additionally to any of the examples above, the core and the stabilizing member may be a unitary component and the device coupling member extends over a portion of the unitary component.

Alternatively or additionally to any of the examples above, the stabilizing member may comprise a strap and a strap connector, and the strap is configured to extend around an umbilicus of the endoscope and couple to the strap connector.

Alternatively or additionally to any of the examples above, the stabilizing member may comprise a slot extending between an outer surface of the device coupling member and the second port, and the slot facilitates engaging the second port with the second fluid port via rotation of the device coupling member.

Alternatively or additionally to any of the examples above, the stabilizing member may comprise an actuator and a retention component in communication with the actuator, and the retention component may be in a first configuration relative to the first port when the actuator is in a first position and the retention component is in a second configuration relative to the first port when the actuator is in a second position.

Alternatively or additionally to any of the examples above, the stabilizing member may comprise a rigid outer member and engaging members extending from the device coupling member, and the first port may be configured to extend around the first fluid port of the endoscope and the rigid outer member is configure to engage the locking members to secure the device coupling member to the endoscope.

In another example, a connector for connecting tubing to fluid ports of a medical device may comprise a first end configured to couple with the tubing and including a first lumen and a second lumen, the second lumen is coaxial with the first lumen, a second end including a first port in fluid communication with the first lumen and configured to engage a first fluid port of the medical device, and a second port in fluid communication with the second lumen and configured to engage a second fluid port of the medical device, and a stabilizing component configured to facilitate securing the second end to the medical device when the first port engages the first fluid port.

Alternatively or additionally to any of the examples above, the stabilizing component may be configured as a ring in the first port and the ring is configured to center the first fluid port within the first port and the second fluid port in the second port.

Alternatively or additionally to any of the examples above, the ring may be formed from a rigid material and the first port and the second port are at least partially formed from a resilient material.

Alternatively or additionally to any of the examples above, the first lumen and the second fluid lumen may form an elbow between the first end and the second end.

Alternatively or additionally to any of the examples above, the stabilizing component may comprise a tab extending transverse to an axis of the first lumen at the first end.

Alternatively or additionally to any of the examples above, the stabilizing component may comprise an elbow joint between the first end and the second end.

Alternatively or additionally to any of the examples above, the first end may be formed from a rigid material, the second end is formed from a resilient material, and the resilient material overlaps the rigid material.

Alternatively or additionally to any of the examples above, the stabilizing component may comprise a retention component configured to engage a component of an endoscope system.

In another example, a connector for connecting tubing to fluid ports of a medical device may comprise a first end configured to couple with the tubing and including a first lumen and a second lumen, the second lumen is coaxial with the first lumen, a second end including a first port in fluid communication with the first lumen and configured to engage a first fluid port of the medical device, and a second port in fluid communication with the second lumen and configured to engage a second fluid port of the medical device, and an actuator, a retention component in communication with the actuator, and the retention component may be configured to adjust to a fluid port engaging position in the first port in response to a first actuation of the actuator and adjust to a fluid port disengaged position in response to a second actuation of the actuator.

Alternatively or additionally to any of the examples above, the retention component may be biased to the fluid port engaging position.

Alternatively or additionally to any of the examples above, a material forming the second end may bias the retention component to the fluid port engaging position.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a schematic side view of an illustrative connector;

FIG. 4 depicts a schematic cross-section view of an illustrative connector;

FIG. 5 depicts a schematic perspective view of an illustrative connector;

FIG. 6 depicts a schematic end view of the illustrative connector depicted in FIG. 5;

FIG. 7 depicts a schematic cross-section view of the illustrative connector, taken along line 7-7 in FIG. 6;

FIG. 8 depicts a schematic perspective view of an illustrative stabilizing member of a core component for a connector;

FIG. 9 depicts a schematic view of the connector with an illustrative stabilizing member;

FIG. 10 depicts a schematic cross section view of the connector depicted in FIG. 9, taken along line 10-10;

FIG. 11 depicts a schematic view of an illustrative connector;

FIGS. 12A-12C depict schematic cross-section views of an illustrative connector coupling with a fluid port of an endoscope;

FIG. 13 depicts a schematic cross-section view of an illustrative connector;

FIG. 14 depicts a schematic cross-section view of an illustrative connector;

FIG. 15 depicts a schematic perspective view of an illustrative connector;

FIGS. 16A and 16B depict the illustrative connector of FIG. 15 adjusting between a first position and a second position;

FIGS. 17A and 17B depict schematic views of an illustrative connector in a first position and in a second position;

FIG. 18 depicts a schematic perspective view of an illustrative connector; and

FIG. 19 depicts a schematic cross-section view of the connector of FIG. 18, taken along line 19-19.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

An endoscope is used in performing diagnostic and/or therapeutic treatments by inserting an elongated shaft of the endoscope into a subject to observe a part to be examined within a body cavity of the subject and, if necessary, inserting a treatment instrument/tool into a working channel in the elongated shaft of the endoscope. Such endoscopes or endoscope systems may include a fluid/lens wash capability, or the like, configured to feed fluid such as gas (e.g., air, CO₂) to an end of the endoscope for insufflating the inside of the subject at a target site. Lens wash features may provide sterilized water at relatively high pressure to spray across and clear debris from a camera lens of the endoscope. In order to rinse the target site of the subject, separate from the air/water feed capability, endoscopes or endoscope systems may have an irrigation capability that provides lower pressure, higher volume water, supplied via a pump (e.g., peristaltic pump) to the target site in order to clear the field of view for observation and treatment. A water source for lens wash and/or irrigation features may include one or more fluid reservoirs having tubing and cap assemblies that create a plumbing circuit in connection with the endoscope channels, valving, and/or connectors to accomplish the gas and water functions described.

Such tubing and cap assemblies may be available in various configurations, which may include a water bottle, a cap fitted for the specific bottle, and an array of tubing that is extendable through openings in the cap. The tubing typically is arranged to accommodate a specific configuration of endoscope fittings and valving, which does not tend to be modular or optional.

When an endoscope procedure utilizing lens wash, irrigation, and/or insufflation features is complete, the endoscope is replaced with a new endoscope and the tubing assemblies may be coupled to the new endoscope via a connector between the tubes for the lens wash, irrigation, and/or insufflation features and the endoscope (e.g., an umbilicus of the endoscope). In some cases, the connector may have a rubberized feature that connects to the endoscope via a friction fit over a fluid port of the endoscope. In some cases, the connector can leak while attached to the endoscope due to wear, manufacturing tolerances, and/or other reasons. Further, as the connector may extend from the endoscope in a manner that is horizontal and/or parallel with or substantially parallel with a floor surface, stress may be applied to the connector due to gravitational forces acting on the horizontally extending connector. This disclosure discusses various connectors and connector and/or tubing assemblies that address the above noted issues and/or other issues with existing connectors.

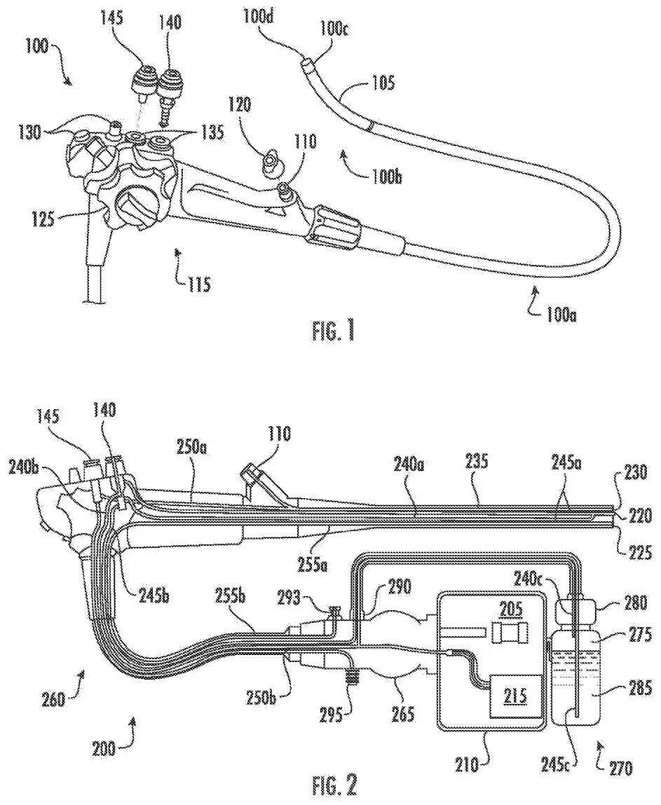

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient). Details of the endoscope 100 and the endoscope system 200 may be more fully described in U.S. Patent Application Publication No. 2022/0192479 A1, filed on Dec. 21, 2021, and titled TUBING ASSEMBLIES AND METHODS FOR FLUID DELIVERY, which is hereby incorporated by reference in its entirety for all purposes.

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., a lamp) may be located in a video processing unit 210 that processes signals input from the imager and outputs processed video signals to a video monitor (not shown) for viewing. The video processing unit 210 may also serve as a component of an air/water feed circuit by housing a pressurizing pump 215, such as an air feed pump, in the unit 210. Other suitable pumps for the air/water feed circuit are contemplated.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the subject at the treatment area and for supplying water to wash a lens covering the imager. An irrigation opening 225 in the end face 100d supplies irrigation fluid to the treatment area of the subject. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob may control up-down steering and another knob may control left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 may have a gas (e.g., air or CO₂) feed line 240b, a lens wash feed line 245b, a suction feed line 250b, an irrigation feed line 255b, a light guide (not shown), an electrical signal cable (not shown), and/or other suitable lines, guides, and/or cables. The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265, when plugged into the video processing unit 210, also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c may pass from one end positioned in an air gap 275 between the top 280 (e.g., a bottle cap) of the reservoir 270 and the remaining water 285 in the reservoir to a connector 290 on the outside of the connector portion 265. The gas feed line 240b from the umbilical 260 branches in the connector portion 265 to fluidly communicate with the gas supply tubing 240c at the detachable connector 290, as well as the air pump 215. A length of lens wash tubing 245c, with one end positioned at the bottom of the reservoir 270, may pass through the top 280 of the reservoir 270 to the same detachable connector 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some configurations, irrigation water may be supplied via a pump (e.g., peristaltic pump) from a water source (not shown) independent from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

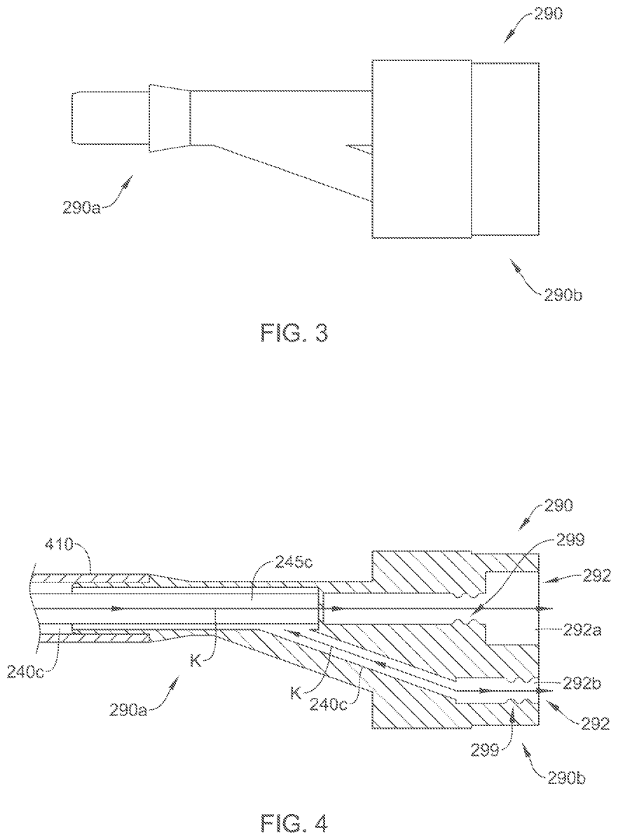

The gas supply tubing 240c and the lens wash tubing 245c may be combined in a coaxial relationship, but this is not required. In one example, the gas supply tubing 240c may define a lumen that is sufficiently large in diameter to encompass a smaller diameter lens wash tubing 245c, coaxially received within the gas supply tubing, as well as provide air to the water source in an annular space surrounding the lens wash tubing to pressurize the water reservoir (e.g., gas tubing 240c and lens wash supply tubing 245c as configured in the connector 290 depicted in FIGS. 3 and 4). The lens wash supply tubing 245c may be configured to exit the lumen defined by the coaxial gas supply tubing in any suitable sealed manner, such as for example an aperture, fitting, collar, and/or the link for the purpose of transitioning from the coaxial arrangement to a side-by-side arrangement of the detachable gas/lens wash connection to the endoscope connector portion 265. In one example, a fitting for such a transition is depicted as the connector 290 in FIGS. 3 and 4 (e.g., a coaxial split connector, but other suitable connector configurations are contemplated).

Referring to FIGS. 3 and 4, in one configuration, the connector 290 may be in the form of a coaxial split connector configured to couple to the coaxial tubing 410, either directly or indirectly, at a first end 290a (e.g., a proximal end) and the umbilical 260 (e.g., a connector 265 of the umbilical 260) of the endoscope system 200 at a second end 290b (e.g., a distal end). For example, in connection with the illustrated configuration in FIG. 4, the connector 290 may be coupled, directly or indirectly, to the endoscope end (e.g., a distal end) of coaxial tubing 410, so that the coaxial portions of the tubing 410 may be coupled to the endoscope 100 via the connector 290. In use, the connector 290 may allow for flow to transition between a coaxial arrangement and a side-by-side arrangement with flow paths K through ports 292 (e.g., a first port 292a for water and a second port 292b for gas), shown in FIG. 4. In some cases, the first port 292a and the second port 292b may be parallel to one another, but this is not required and other suitable relative positions of the first port 292a and the second port 292b are contemplated. Although the connector 290 is depicted as a coaxial split connector in FIG. 4, other suitable connectors 290 may be configured to connect parallel tubing or a single tube to the umbilical 260 of the endoscope system 200, as desired.

In some cases, the ports 292 may include one or more features configured to facilitate the connector 290 engaging a fluid port of the endoscope system 200. As depicted in FIG. 4, the ports 292 may include features such as coupling portions 299 (e.g., ribs, ridges, etc.) configured to engage a male feature of the fluid ports of the endoscope system. Alternatively or additionally, the one or more features configured to engage a fluid port of the endoscope system 200 may take on one or more other suitable configurations including, but not limited to, a one-way valve, a duck-bill valve, and/or other suitable features configured to engage the fluid ports of the endoscope system 200.

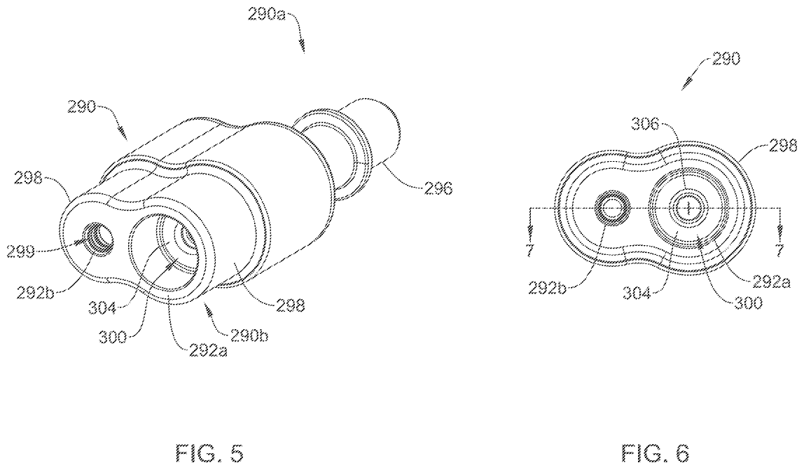

The connectors 290 depicted in FIGS. 5-19 are configured to improve upon current gas (e.g., air, etc.) and liquid (e.g., water) connectors for endoscope systems. For example, disclosed connectors 290 may be configured to prevent or mitigate leakage at a connection between tubing and an umbilical of the endoscope system, reduce stress on tubing extending proximal from the connector 290, improve ergonomics of the connector 290 related to the plugging and unplugging of the connector 290 to an endoscope umbilical, reduce potential for cross contamination when the connector 290 is used for multiple procedures on different subjects and/or configured to provide one or more other suitable benefits including, but not limited to, those discussed herein.

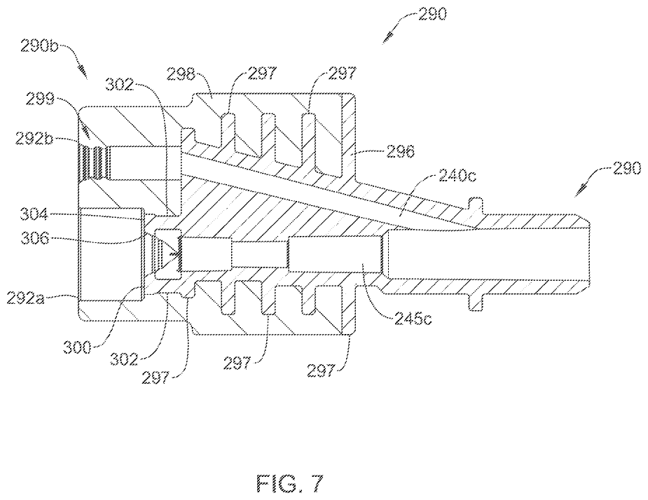

FIGS. 5-7 depict schematic views of an illustrative connector 290 with a core 296, a device coupling member 298, and a stabilizing member 300. FIG. 5 is a schematic perspective view of the connector 290. FIG. 6 is a schematic end view of the connector 290 depicted in FIG. 5. FIG. 7 is schematic cross-section view of the connector 290, taken along line 7-7 in FIG. 6.

In some cases, a notable amount of force may be required to couple the ports 292 of the connector 290 to fluid ports of an umbilical via a friction fit or other suitable type of connection and thus, if the connector is not centered properly relative to the fluid ports of the umbilical, the connector 290 may incur damage at or around the ports 292. In some cases, damage at or around the ports 292 may lead to fluid leaking along the ports 292. The connector 290 depicted in FIGS. 5-7 may include a stabilizing member 300 in the first port 292a that is configured to facilitate centering the first port 292a about a first fluid port of an umbilical and prevent the device coupling member 298 of the connector 290 from skiving and/or otherwise deforming due to forces needed to couple the first fluid port to the first port 292a. Further, the stabilizing member 300 centering the first port 292a about the first fluid port (or centering the first fluid port within the first port 292a) may also result in facilitating centering the second fluid port within or otherwise relative to the second port 292b.

In some case, the stabilizing member 300 may be ring-shaped and may form a proximal end of and/or a support structure the first port 292a. The ring-shaped stabilizing member 300 may be configured to facilitate centering the first port 292a relative to and engaging or securing the first port 292a with or to the first fluid port of the umbilical. The ring-shaped stabilizing member 300 may support a shape of the first port 292a by providing a rigid support structure to the first port 292a.

The schematic cross-section view of FIG. 7 depicts the connector 290 with the core 296, the device coupling member 298 extending over the core 296 (e.g., where the material of the device coupling member 298 may overlap the material of the core 296) and the stabilizing member 300, which may extend from the core 296. In some cases, the core 296 may be part of or may be unitarily or monolithically formed with the stabilizing member 300 (e.g., the core 296 and the stabilizing member 300 may be a unitary component), but this is not required and the core 296 and the stabilizing member 300 may be separately formed.

The core 296 may be configured to provide a core or supporting structure to the connector 290 and provide a mechanism for separating lumens of the coaxial tubing coupled to a proximal or first end 290a of the connector 290. Although not required, the core 296 may extend from the first end 290a of the connector 290 to a proximal end of the ports 292.

In some cases, the core 296 may have one or more ribs 297 extending radially outward from portions of the core 296 defining the lumens or tubing 240c and 245c to provide support to the material of the device coupling member 298. The distal-most ribs 297 of the core 296 may form rigid proximal surfaces and/or supports for the ports 292. The proximal most ribs 297 may form a proximal support surface that stabilizes the connector 290 as it is connected with fluid ports of the umbilical. Additionally or alternatively, the proximal-most ribs 297 may provide a surface on which the device coupling member 298 may be formed on the core 296.

The stabilizing member 300 may extend distally from the core 296 and into the first port 292a. Although other suitable configurations are contemplated, the stabilizing member 300 may include one or more posts 302 and a ring 304. In some cases, one or more of the posts 302 may extend distally from the core 296 and into the first port 292a of the connector 290. The posts may support the ring 304 within the first port 292a.

As depicted in FIG. 7, an inner circumference of the ring 304 may have a tapered surface 306 tapering toward a central axis in a direction of insertion of a fluid port of an umbilical into the first port 292a. The tapered surface 306 may facilitate centering the first port 292a about the first fluid port of the umbilical, which may facilitate the centering the second port 292b about the second fluid port of the umbilical.

The core 296 and the stabilizing member 300 depicted in FIGS. 5-7 may be formed from any suitable materials. In some cases, the one or more materials of the core 296 and/or the stabilizing member 300 may be more rigid relative to a rigidity of a material of the device coupling member 298. Example suitable materials for the core 296 and/or the stabilizing member 300 of FIGS. 5-7 may include, but are not limited to, one or more of metals, polymers, composites, liquid crystal polymers, acrylonitrile butadiene styrene (ABS), polypropylene (PP), polyvinyl chloride (PVC), polycarbonate, and/or other suitable rigid materials. In one example, the core 296 may be formed from polycarbonate, but this is not required.

The device coupling member 298 may entirely or at least partially define the ports 292 of the connector 290. In some cases, the device coupling member 298 may define one or more of the ports 292 with the core 296 and/or the stabilizing member 300 (e.g., as depicted in FIGS. 5-7), but this is not required.

The device coupling member 298 may be formed from any suitable elastic or resilient material configured to create a friction fit with the fluid ports of the umbilical. Example elastic and/or resilient materials suitable for forming the device coupling member 298 include, but are not limited to, one or more of polymer, composites, rubber, elastomers, silicone, polyether-amide (PEBA) block copolymers (e.g., PEBAX or other suitable PEBA block copolymers), ethylene propylene diene monomer (EPDM) rubber, thermoplastic elastomers (TPE), and/or other suitable materials. In one example, the device coupling member 298 may be formed from TPE, but this is not required.

The device coupling member 298 may be configured to couple to the core 296 and/or the stabilizing member 300 in any suitable manner. In some cases, the device coupling member 298 may be over-molded onto the core 296 and/or the stabilizing member 300, but this is not required and the device coupling member 298 may be coupled to the core 296 and/or the stabilizing member 300 in one or more other suitable manners.

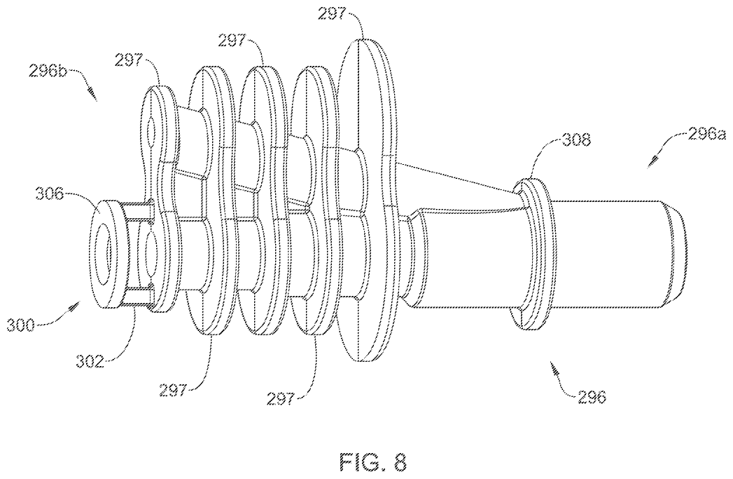

FIG. 8 depicts a schematic view of the core 296 and the stabilizer member 300 extending distally from the core 296. Although not required, a proximal-most rib 297 of the core 296 may have a larger surface area than the other ribs 297 and may be configured to support the device coupling member 298, as discussed above. The distal-most rib 297 may have a smaller surface area than the other ribs 297 and may be configured to form proximal ends of the ports 292, but this is not required, as discussed above. In some cases, core 296 may extend proximally from the distal most rib 297 and may be configured to form a proximal end of the first port 292a.

The core 296 may have a first or proximal end 296a and a second or distal end 296b. The proximal end 296a of the core 296 may be configured to receive coaxial tubing, where the tubing may engage the proximal end 296a and extend onto the core 296 until it reaches a stop 308, but this is not required and the tubing may engage the core 296 and/or the connector 290 in one or more other suitable manners.

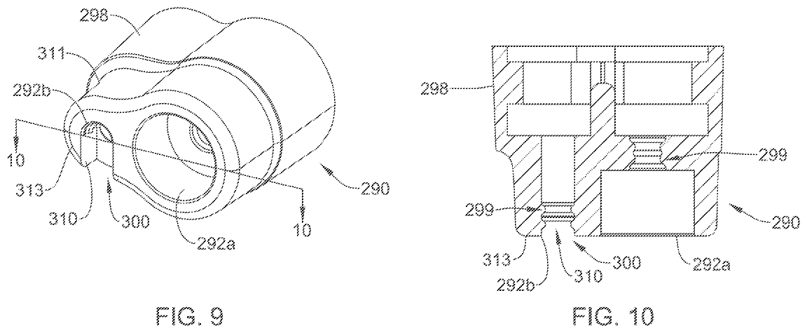

An illustrative configuration of a connector 290 having a stabilizing feature or member 300 in the device coupling member 298 is depicted in FIGS. 9 and 10. Similar to as discussed above with respect to the connector 290 of FIGS. 5-7, the device coupling member 298 may at least partially define ports 292 and may be formed from an elastic material configured to create a friction fit with fluid ports of an umbilicus. A core is omitted from the connector 290 depicted in FIGS. 9 and 10, but this is not required and a core 296 similar to that depicted in FIG. 8 and/or other suitable cores may be utilized to support the device coupling member 298.

As depicted in FIG. 9, the stabilizing member 300 of the connector 290 may include a slot 310 extending radially inward from an outer side surface 311 of the device coupling member 298 to the second port 292b (e.g., a gas port or other suitable port) and extending proximally inward from an end surface 313. The configuration of the slot 310 may facilitate the second port 292b initially receiving a fluid port of the umbilical in response to rotation of the connector 290.

The slot 310 may have any suitable depth. In some cases, the slot 310 may have a depth sufficient to allow the first port 292a to receive and center on the first fluid port of the umbilical, yet still allow the second fluid port of the umbilical to subsequently engage the second port 292b through the slot 310.

In operation, a first fluid port of the umbilical may be inserted into the first port 292a of the connector 290 without the second port 292b necessarily aligned with a second fluid port of the umbilical. Once the first fluid port is partially inserted into and centered in the first port 292a of the connector 290, the connector 290 may be rotated and as the connector 290 is rotating the second fluid port may be received in the slot 310 and the second port 292b. In some cases, the connector 290 may be rotated in a clockwise direction to engage the second fluid port, but this is not required. Once the connector 290 has been rotated to a position at which the second port 292b is contacting the second fluid port of the umbilical, the connector 290 may be advanced further (e.g., distally) over the first and second fluid ports to fully connect the connector 290 to the umbilicus such that the coupling portions 299 engage external coupling portions on the fluid ports. In some cases, the coupling portions 299 may be omitted.

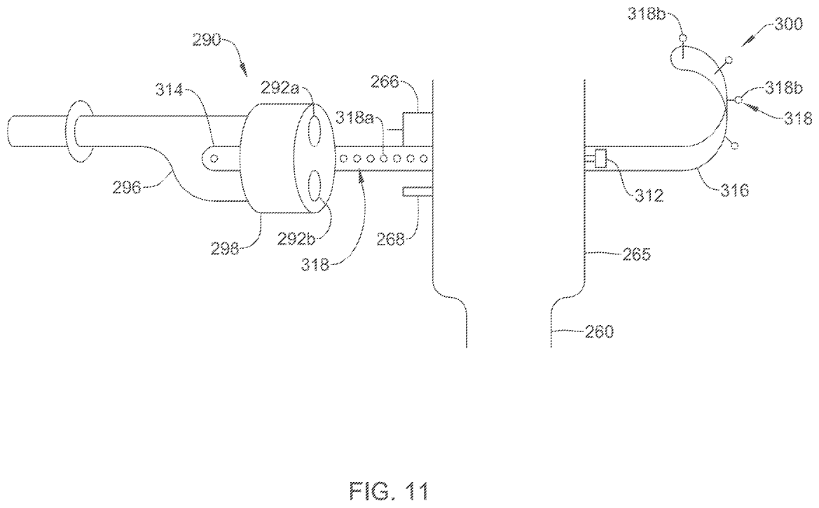

FIG. 11 depicts an illustrative configuration of a connector 290 having a stabilizing feature or member 300 that is configured to wrap around and engage a first locking feature 312 (e.g., a first strap connector) on the connector portion 265 of the umbilical 260 and/or a second locking feature 314 (e.g., a second strap connector) on the core 296 and/or the device coupling member 298. The stabilizing member 300 may take on the form of a belt or strap 316 and may extend from one or both of the core 296 and the device coupling member 298 of the connector 290, as desired.

The strap 316 may include one or more lock engaging features 318 that are configured to engage one or both of the first locking feature 312 and the second locking feature 314. The lock engaging features 318 may be any suitable features configured to engage one or both of the first locking feature 312 and the second locking feature 314 and may include, but are not limited to, holes, tabs, buttons, snaps, clasps, prongs, and/or other suitable lock engaging features. In one example, a first lock engaging feature 318a may include holes configured to engage the first locking feature 312 (e.g., a post, prong, and/or other suitable locking feature) and a second lock engaging feature 318b may include prongs configured to engage the second locking feature 314 (e.g., a hole, an opening, loop, and/or other suitable locking feature), but other configurations of the locking features 312, 314 and the lock engaging features 318 are contemplated. Although not required the connector 290 and/or the connector 265 of the umbilical 260 may include one or more buckles or openings through which the strap 316 may be threaded.

In operation, a user may engage the first port 292a with a first fluid port 266 of (e.g., extending from) the connector 265 of the umbilical 260 and the second port 292b with a second fluid port 268 of (e.g., extending from) the connector 265. Once engaged, the strap 316 may be extended around the connector 265 of the umbilical 260, the first lock engaging features 318a may engage the first locking feature 312, the strap 316 may be further extended around the connector 265 and passed back to the core 296 and/or the device coupling component 298, and the second lock engaging features 318b may engage the second locking feature 314. Such a configuration ensures the connector 290 is pressed against the connector 265 during use and prevents the connector 290 from disengaging from the umbilical 260 and reduces forces acting on the device coupling member 298 caused by gravity and tubing extending proximally from the connector 290. Further, when the strap 316 is utilized as the stabilizing member 300, the first and second ports 292 may be designed to have less interference and/or friction with the fluid ports 266, 268 of the umbilical 260, which may allow for the device coupling member 298 to more easily engage the fluid ports 266, 268 than when a tight friction fit is required for connecting the connector 290 to the fluid ports 266, 268.

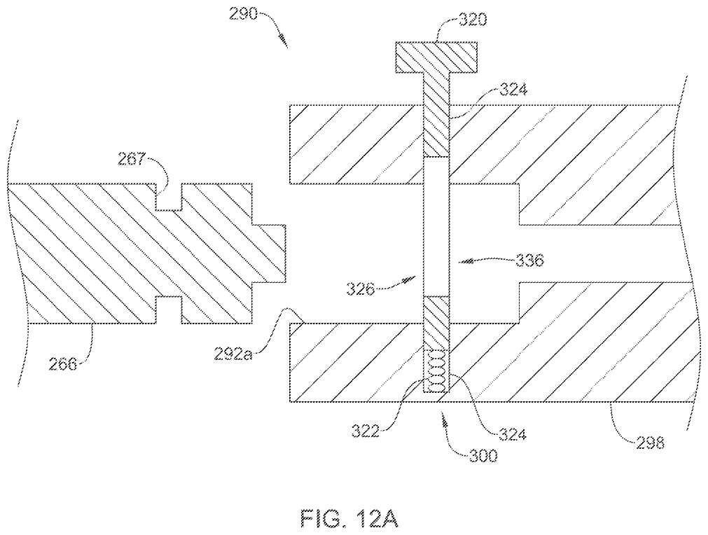

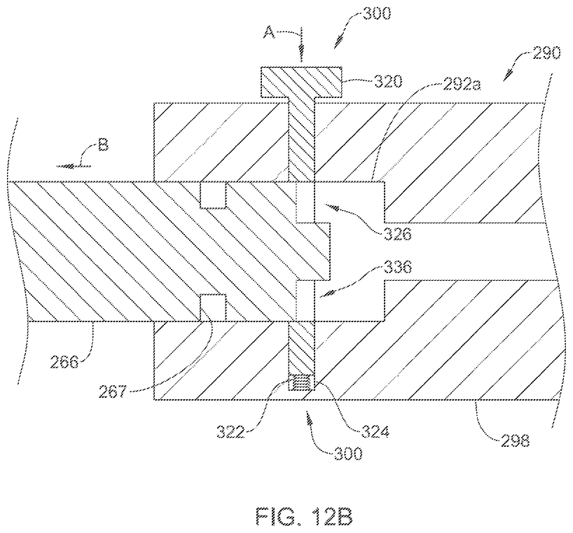

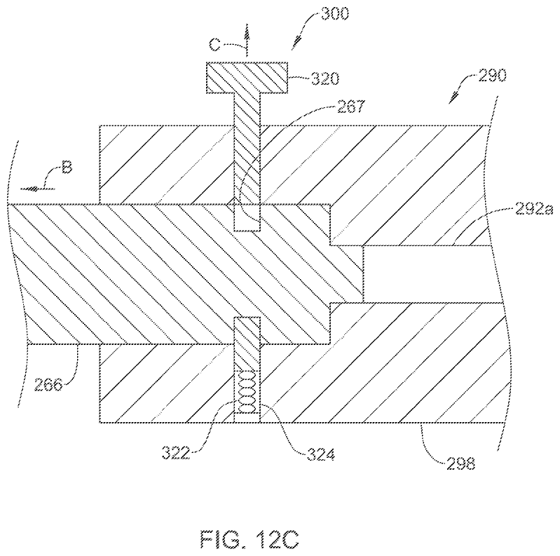

In some cases, configurations of the connector 290 may include an actuatable stabilizing member 300 that may be configured to adjust relative to the device coupling member 298. In one example, the stabilizing member 300 may be adjusted to facilitate receiving a fluid port from an endoscope umbilical and further adjusted to facilitate engaging the fluid port. FIGS. 12A-12C depict schematic cross-section illustrations of a connector 290 that includes a stabilizing member 300 that may be actuated to facilitate receiving, engaging, and disengaging one or more fluid ports of an endoscope. Although the connector 290 is depicted in FIGS. 12A-12C without a core, it is contemplated that the connector 290 depicted in FIGS. 12A-12C may include a core configured similar to or different than the core 296.

The configuration of the connector 290 with a stabilizing member 300 depicted in FIGS. 12A-12C may be configured to be adjusted to receive, engage, and disengage a fluid port (e.g., the first fluid port 266 and/or other suitable fluid port having an engaging feature 267) of an endoscope umbilical. In some cases, the stabilizing member 300 may include an actuator 320 and a biasing member 322. Although FIGS. 12A-12C depict the stabilizing member 300 in the first port 292a and configured to receive the first fluid port 266, the stabilizing member 300 may be located in other ports of the connector 290 and configured to receive other fluid ports of an umbilicus of an endoscope, as desired.

The actuator 320 may be configured to extend through a slot 324 leading to and/or or through the port(s) 292 (e.g., the first port 292a and/or other suitable port) of the connector 290 from exterior of the device coupling member 298. Alternatively or additionally, an actuatable portion of the actuator 320 may be embedded within material of the device coupling member 298, where the material of the device coupling member 298 is configured such that the actuator 320 may be actuated through the material.

The actuator 320 may include an opening 326 configured to receive the fluid port of the umbilical. In some cases, the opening 326 may be or may be in a retention component 336 of or coupled with the actuator 320, such that the retention component 336 may adjust positions in response to actuation of the actuator 320.

The opening 326 may be any suitable shape and/or size configured to receive the fluid port when the actuator 320 is in an actuated position and engage the fluid port when the actuator 320 is in a resting position. In one example, the opening 326 may be circular or substantially circular and sized to receive a fluid port, but this is not required.

The biasing member 322 may be located in the slot 324 proximate the port 292a of the connector 290. The biasing member 322 may be any suitable type of biasing member configured to facilitate biasing the actuator 320 and/or the retention component 336 to a resting or engaging position and movement of the actuator 320 from the resting or engaging position to an actuated position for receiving a fluid port in and/or disengaging a received fluid port from the stabilizing member 300. In one example, the biasing mechanism 322 may be a spring in the slot 324, as shown in FIGS. 12A-12C. In another example, the biasing mechanism 322 may be a material of the device coupling member 298 that has a spring constant configured to facilitate the movement of the actuator 320 and/or the retention component 336 discussed herein. In a further example, the biasing mechanism 322 may be a material inserted into the slot 324 that has a spring constant configured to facilitate the movement of the actuator 320 discussed herein, where the material inserted into the slot 324 may be the same or different than the material of the device coupling member 298 defining the slot 324. Other suitable configurations of the biasing mechanism 322 are contemplated.

FIGS. 12A-12C depict a process for coupling the connector 290 with the first fluid port 266. FIG. 12A depicts the actuator 320 and the retention component 336 of the stabilizing member 300 in a first position (e.g., a resting or biased position) that may be configured to engage a fluid port of an endoscope.

FIG. 12B depicts the actuator 320 and/or the retention component 336 actuated to a second position (e.g., a receiving or actuated position) in the direction of arrow A such that the first fluid port 266 may be inserted into the first port 292a by adjusting the connector 290 in the direction of arrow B and advancing the first fluid port 266 through the opening 326 of the actuator 320. When the actuator 320 is actuated in the direction of arrow A, the biasing mechanism 322 may be compressed.

FIG. 12C depicts the first fluid port 266 fully inserted into the first port 292a through the opening 326 of the actuator 320 and/or the retention component 336 such that the engaging feature 267 may be aligned with the opening 326. When a force in the direction of arrow A and acting against the biasing mechanism 322 is removed from the actuator 320, the actuator 320 may be adjusted (e.g., biased) in a direction of arrow C and may engage (e.g., clip to and/or otherwise engage) the engaging feature 267 of the first fluid port 266. In some cases, the biasing mechanism 322 may automatically adjust the actuator 320 in the direction of arrow C when the force in the direction of arrow A is removed from the actuator 320, but this is not required. To disengage the connector 290 from the first fluid port, the steps in FIGS. 12A-12C may be reversed.

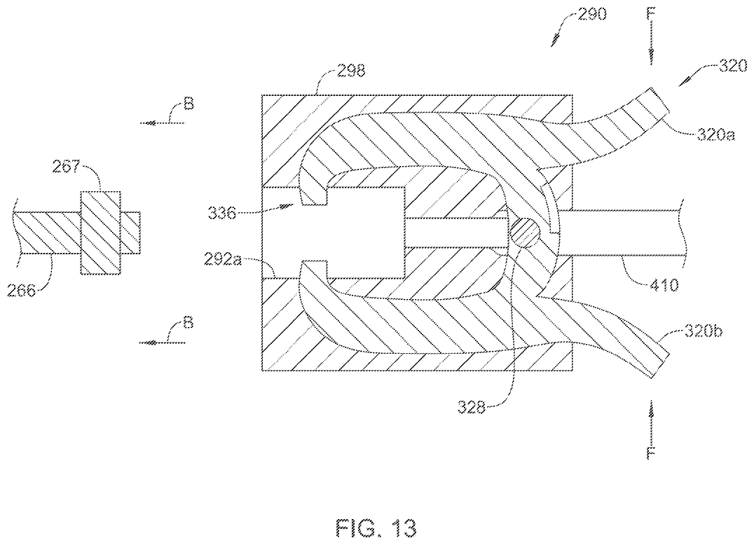

FIG. 13 depicts a schematic cross-section illustration of a connector 290 that includes a stabilizing member 300 that may be actuated to facilitate receiving, engaging, and disengaging one or more fluid ports of an endoscope. Although the connector 290 is depicted in FIG. 13 without a core, it is contemplated that the connector 290 depicted in FIG. 13 may include a core configured similar to or different than the core 296.

The configuration of the connector 290 with a stabilizing member 300 depicted in FIG. 13 may be configured to be adjusted to receive, engage, and disengage a fluid port from an umbilical of an endoscope (e.g., the first fluid port 266 and/or other suitable fluid ports having an engaging feature 267, such as a protrusion as depicted in FIG. 13, a slot as depicted in FIGS. 12A-12C, and/or other suitable configuration). In some cases, the stabilizing member 300 may include an actuator 320 having a first component 320a and a second component 320b, where the first and the second components 320a, 320b are configured to pivot about a pin 328. In some cases, a distal end of the actuator 320 (e.g., distal ends of the first and second components 320a, 320b) may form a retention component(s) 336 configured to engage one or more fluid ports of an endoscope.

In the configuration of the connector 290 depicted in the FIG. 13, material of the device coupling component 298 may be resilient and may have a spring constant, such that the material may act as a biasing mechanism on the actuator 320 and bias the actuator 320 and/or the retention component(s) 336 to a resting position depicted in FIG. 13 configured to engage a fluid port of an umbilical. Alternatively or additionally, one or more biasing mechanisms (e.g., a spring and/or other suitable biasing mechanism(s)) may be utilized to bias the first actuator component 320a and the second actuator component 320b to the resting position.

In operation, a user may engage the first and second actuator components 320a, 320b and apply a force in the directions of arrows F on the respective actuator components 320a, 320b (e.g., apply a pinching or squeezing force or motion to the actuator components 320a, 320b and/or other suitable motion) to adjust the actuator 320 and/or the retention components 336 from a first position (e.g., closed or resting position) to a second position (e.g., opened, receiving or engaging position). When in the second position, the connector 290 may be moved in the direction of arrow B toward the first fluid port 266 until the engaging feature 267 is within the first port 292a such that actuator 320 and/or the retention components 336 may clasp or otherwise couple to the engaging feature 267 of the first fluid port 266. When the first fluid port 266 has been fully inserted into the first port 292a and the actuator 320, the force in the direction of arrows F may be removed and the actuator 320 and/or the retention components 336 may be biased to the first position so as to engage the engaging feature 267 of the first fluid port 266. Although FIG. 13 depicts the stabilizing member 300 in the first port 292a and may be configured to receive the first fluid port 266, the stabilizing member 300 may be located in other ports of the connector 290 and configured to receive other fluid ports of an endoscope umbilical, as desired.

The material of the device coupling member 298 of the connector 290 depicted in FIG. 13 may be any suitable type of material configured to bias the actuator 320 to a resting position and minimize or reduce a likelihood of the actuator 320 releasing the fluid port in an unintended manner. Example suitable materials for the device coupling member 298 include, but are not limited to, one or more of materials that are flexible, materials that provide sealing capabilities, elastomeric materials, EPDM, silicone, PEBA block copolymers, and/or other suitable materials.

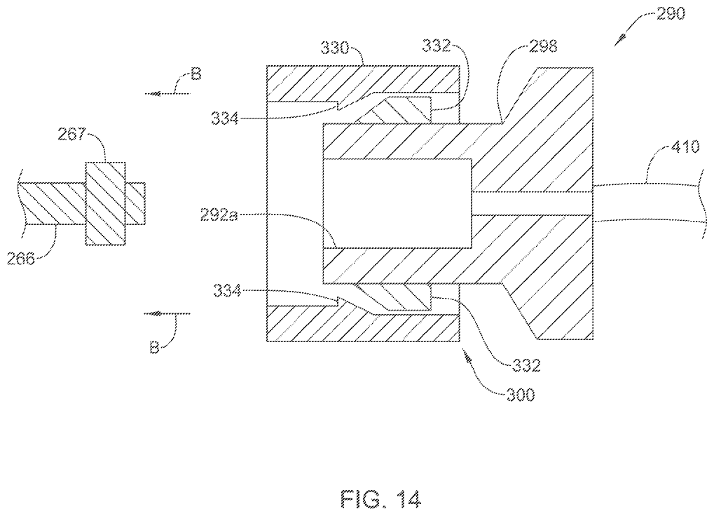

The material of the actuator 320 and/or the retention component(s) 336 of the connector 290 depicted in FIG. 13 may be any suitable type of material configured to be biased by the material of the device coupling member 298 and/or other suitable biasing mechanism. Example suitable materials for the actuator 320 and/or the retention component 336 may include, but are not limited to, one or more of ABS, PP, PVC, polycarbonate, and/or other suitable materials. In one example, the actuator 320 and/or the retention component(s) 336 may be formed from PVC, but this is not required. FIG. 14 depicts a schematic cross-section illustration of a connector 290 that includes a stabilizing member 300 that may be actuated to create a secure engagement between the connector 290 and one or more fluid ports of an endoscope. Although the connector 290 is depicted in FIG. 14 without a core, it is contemplated that the connector 290 depicted in FIG. 14 may include a core configured similar to or different than the core 296.

The configuration of the connector 290 with a stabilizing member 300 depicted in FIG. 14 may be configured to be adjusted to receive, engage, and disengage a fluid port (e.g., the first fluid port 266 and/or other suitable fluid port having an engaging feature 267, such as a protrusion as depicted in FIG. 14 and/or other suitable configuration) of an endoscope umbilical. In some cases, the stabilizing member 300 may include an outer member or component 330, one or more first engaging members or features 332 (e.g., barbs or extensions) extending from the device coupling member 298, and one or more second engaging features or components 334 extending radially inward from an inner surface of the outer component 330.

In the configuration depicted in FIG. 14, material of the device coupling component 298 may be elastic and/or resilient, such that the material may flex around and engage first fluid port 266 and the engaging feature 267 as the first port 292a of the device coupling member 298 is moved in the direction of arrows B and extends over first fluid port 266. In some cases, the tapered surface of first engaging features 332 may engage a tapered surface of the second engaging features 334 to push the material of the device coupling member 298 tightly around the first fluid port in response to engaging the first engaging features 332, but other suitable configurations are contemplated.

The first engaging features 332 may extend entirely around an outer circumference of the device coupling member 298 and/or one or more first engaging features 332 may be spaced from one another along the outer circumference of the device coupling member 298 (e.g., two spaced apart first engaging features 332, as depicted in FIG. 14). The first engaging features 332 may have a tapered surface that tapers inward in the direction of arrows B and a shouldered surface proximal of the tapered surface and configured to engage the second engaging features 334 to prevent the device coupling member 298 from backing out of the outer component 330.

The second engaging features 334 may extend entirely around an inner circumference of the outer component 330 and/or one or more second engaging features 334 may be spaced from one another along the inner circumference of the outer component 330 (e.g., two spaced apart second engaging features 334, as depicted in FIG. 14). The second engaging features 334 may have a tapered surface that tapers inward in the direction of arrows B and a shouldered surface distal of the tapered surface and configured to engage the first engaging features 332 to prevent the device coupling member 298 from backing out of the outer component 330.

The outer component 330 may be configured to receive the device coupling member 298 and secure the device coupling member 298 therein through engagement of the first and second engaging features 332, 334. Proximal of the second engaging features 334, the outer component 330 may have an inner diameter of a first distance and distal of the second engaging features 334, the outer component 330 may have an inner diameter of a second distance that is less than the first distance to facilitate compressing the material of the device coupling member 298 against the first fluid port 266 when the shoulders of first and second engaging features 332, 334 are engaged and the first engaging features abut the inner surface of the outer component 330 defining the second distance.

The material of the outer component 330, the first engaging features 332, and/or the second engaging features 334 depicted in FIG. 14 may be any suitable type of material configured to be rigid, yet flexible so as to bend when engaging and/or disengaging the outer component 330 with the device coupling member 298. Example suitable materials for the actuator 320 include, but are not limited to ABS, PP, PVC, polycarbonate, and/or other suitable materials. In one example, the actuator 320 may be formed from PVC, but this is not required.

The material of the device coupling member 298 of the connector 290 depicted in FIG. 14 may be any suitable type of material configured to resiliently flex in response to forces acting thereon. Example suitable materials for the device coupling member 298 include, but are not limited to, elastomeric materials, EPDM, silicone, PEBA block copolymers, and/or other suitable materials.

In operation, a user may advance the outer component 330 and the device coupling member 298 in the direction of arrows B to engage the first fluid port 266. The outer component will contact the connector of the umbilical (not shown in FIG. 14) and the device coupling member 298 may be advanced further in the direction of arrows B around the first fluid port 266. As the device coupling member 298 advances, the first engaging features 332 (e.g., tapered surfaces of the first engaging features 332 and/or other portions of the first engaging features 332) may extend radially outward from the device coupling member 298 and engage the second engaging features 334 (e.g., tapered surfaces of the second engaging features 334 and/or other portions of the second engaging features 334) extending radially inward from outer component 330 to compress the material of the device coupling component 298 around the engaging feature 267 of the first fluid port 266 to secure the connector 290 to the first fluid port 266.

To release the connector 290 from the first fluid port, a radially inward pinching or squeezing force or other suitable force may be applied to an outer surface of the outer component 330 such that the first and second coupling features may be disengaged and the device coupling member 298 may be removed or otherwise disengaged from the first fluid port 266 by adjusting the device coupling member 298 in a direction opposite of the arrows B. In some cases, when the pinching or squeezing force is applied to the outer component 330, the first and second coupling features may be misaligned to allow withdrawal of the device coupling member 298 from the first fluid port 266 and/or the outer component 330. Although the connection of the connector 290 to a fluid port is depicted in FIG. 14 and described with respect to the first port 292a and configured to couple to the first fluid port 266, the stabilizing member 300 may be located in other ports of the connector 290 and configured to receive other fluid ports of an endoscope umbilical, as desired.

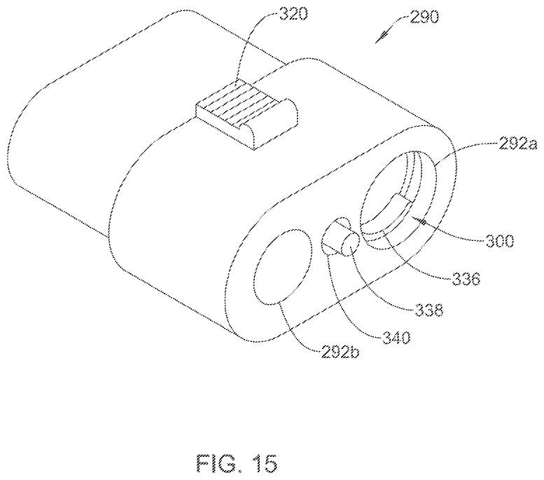

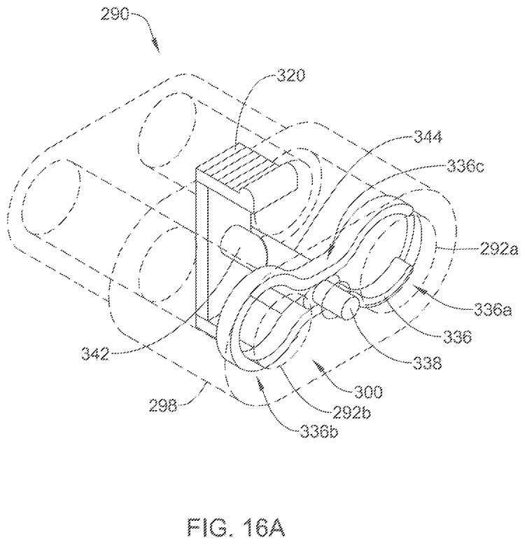

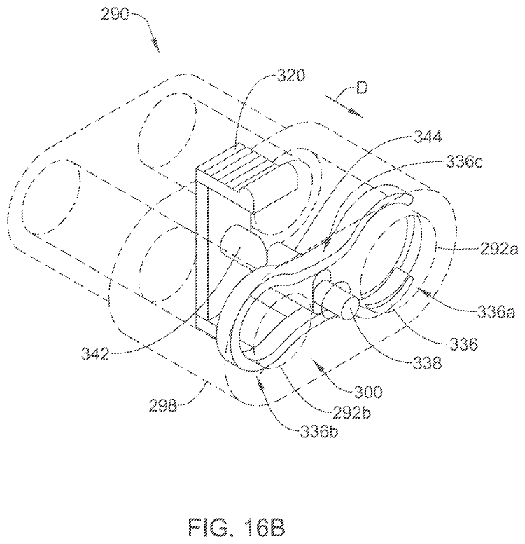

FIGS. 15-16B depict a connector 290 having a stabilizing member 300 with an actuator 320 configured to cause a retention component 336 (e.g., a clip and/or other suitable retention component to adjust between a first position configured to receive a fluid port and a second position configured to engage or couple to the received fluid port. FIG. 15 depicts a schematic perspective view of the connector 290 and FIGS. 16A and 16B depict schematic perspective views of the components of the stabilizing member 300, where the device coupling member 298 is shown in broken lines to provide an environment for the components of stabilizing member 300. Although the connector 290 is depicted in FIGS. 15-16B without a core, it is contemplated that the connector 290 depicted in FIGS. 15-16B may include a core configured similar to or different than the core 296.

The configuration of the stabilizing member 300 depicted in FIGS. 15-16B may include the actuator 320, the retention component 336, and a shaft 338, where the shaft 338 may rotate in response to an actuation of the actuator 320. In some cases, a distal end face (e.g., a surface facing a connector of an umbilical) may include an opening 340 for receiving a distal end of the shaft 338, but this is not required and the shaft 338 may remain within the device coupling member 298. The depicted configuration of the stabilizing member in FIGS. 15-16B may operate similar to a click-actuated pen and/or other similar actuation mechanisms, but this is not required

FIG. 16A depicts the retention component 336 in the second position configured to engage a received fluid port from an endoscope in the first port 292a. In addition to the actuator 320, the retention component 336, and the shaft 338, the configuration of the depicted stabilizing member 300 may include a first thrust component 342 and a second thrust component 344, among other suitable components. In some cases, the first thrust component 342 may be configured to rotate about the shaft 338 and the second thrust component 344 may be configured to rotate with the shaft 338 in response to movement of the first thrust component 342.

The retention component 336 may be any suitable type of retention component 336 configured to adjust between the first position and the second position. As depicted in FIG. 16A, the retention component 336 may have a first portion 336a, a second portion 336b, and an intermediate portion 336c, where the first portion 336a and the second portion 336b may be rounded or may have one or more other suitable configurations configured to receive a fluid port from an endoscope. In some cases, the first portion 336a may be configured to adjust between the first position configured to receive a fluid port and the second position configured to engage the fluid port. Although not required, the first portion 336a may be located in the first port 292a of the connector 290 and the second portion 336b may be located in the second port 292b of the connector 290.

The retention component 336 may be formed from any suitable material configured to have a bias or to be biased. Example suitable materials include, but are not limited to, metals, polymers, composites, alloys, shape memory materials, plastic, stainless steel, nick-titanium alloys (e.g., nitinol), and/or other suitable materials. The retention components 336 may be configured to be biased to one or more positions by a biasing mechanism (e.g., a spring and/or other suitable biasing mechanism) when the retention component 336 is not formed from a shape memory material or is not resilient. In some cases, the retention component 336 may be biased to a fluid port engaging position (e.g., as depicted in FIG. 16A).

The first thrust component 342 may take on any suitable configuration configured to rotate about the shaft 338 in response to actuation of the actuator 320. In some cases, the first thrust component 342 may have a substantially rounded cross-section and a chamfered distal surface or end, which may be configured to engage a chamfered proximal surface or end of the second thrust component 344.

The second thrust component 344 may take on any suitable configuration configured to rotate with the shaft 338 in response to actuation of the actuator 320. In some cases, the second thrust component 344 may have an ovular or elliptical cross-section and a chamfered proximal surface or end, which may be configured to engage the chamfered distal surface or end of the second thrust component 344. The ovular or elliptical cross-sectional shape of the second thrust component may be configured to such that an outer surface of the second thrust component 344 may engage the retention component 336 to adjust the retention component 336 from the first position to the second position in response to actuation of the actuator 320. As depicted in FIG. 16A, the second thrust component 344 has been adjusted such that an outer surface thereof defining a first height is in contact with the intermediate portion 336c of the retention component 336 causing the second portion 336b of the retention component 336 to be in the second position and configured to engage a received fluid port (e.g., engage an engaging feature or other suitable feature of the received fluid port).

FIG. 16B depicts the actuator 320 actuated in the direction of arrow D. Actuation of the actuator 320 may cause the first thrust component 342 to rotate about the shaft 338, such that the distal end of the first thrust component 342 may engage the proximal end of the second thrust component 344 to cause the second thrust component 344 to rotate with the shaft 338 to the position depicted in FIG. 16B. In the position depicted in FIG. 16B, the second thrust component 344 has been adjusted such that an outer surface thereof defining a second height is in contact with the intermediate portion 336c of the retention component 336 causing the second portion 336b of the retention component 336 to be in the first position and configured to receive a fluid port therethrough. The second height may be greater than first height of the second thrust component such that when the surfaces defining the second height of the second thrust component 344 are engaging the intermediate portion 336c of the retention component 336, an opening through the second portion 336b of the retention component 336 is enlarged to receive a fluid port relative to when surfaces defining the first height of the second thrust component 344 are in contact with the intermediate portion 336c. To return the retention member and the stabilizing member to second position for engaging the fluid port, the actuator 320 may be further actuated in the direction of arrow D and/or actuated in one or more other suitable manners. Further, although the second portion 336b of retention component 336 is depicted in FIGS. 16A and 16B as adjusting about the first port 292a, the retention component 336 may be configured to adjust and retain fluid ports in one or more other suitable ports of the connector 290.

The configurations of the connector 290 depicted in FIGS. 12-16B utilizing an actuator 320, first engaging feature 332, second engaging feature 334, and/or retention component 336 may ensure the connector 290 remains engaged with one or more fluid ports of the umbilicus (e.g., of the connector 265 of the umbilical 260) during use and may prevent the connector 290 from disengaging from the umbilical. Further, when the configurations of the stabilizing member 300 in FIGS. 11-16B are utilized individually or in combination, the first and second ports of the connector 290 may be designed to have less interference and/or friction with the fluid ports of the umbilical, which may allow for the device coupling member 298 to more easily engage the fluid ports than when a tight friction fit is required for connecting the connector 290 to the fluid ports.

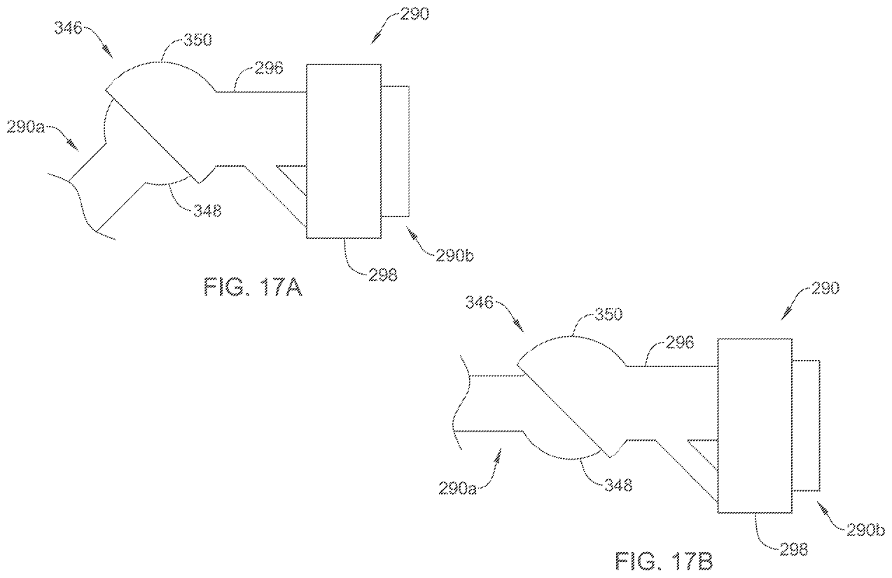

FIGS. 17A and 17B depict a configuration of the connector 290 that includes an elbow joint 346 at a location between a second end 290b where the connector 290 may couple to fluid ports of an endoscope and a first end 290a where the connector 290 may couple to fluid tubing. The lumens of the connector 290 may follow the angle of the joint and thus, the lumens may form an adjustable elbow between the first end 290a of the connector 290 and the second end 290b of the connector 290.

The elbow joint 346 may include a ball component 348 and a socket component 350, but this is not required and other suitable configurations are contemplated for the elbow joint 346. FIG. 17A depicts the ball 348 and the first end 290a of the connector 290 rotated to a first position within or relative to the socket 350. FIG. 17B depicts the ball 348 and the second end 290b of the connector 290 rotated to a second position within or relative to the socket 350. In some cases, when the ball 348 and the first end 290a of the connector 290 are in the first position within or relative to the socket 350, stress on the tubing and the connector may be reduced relative to the when the ball 348 and the first end 290a of the connector 290 are in the second position within or relative to the socket 350 due to the first end 290a of the connector and tubing connected thereto being positioned in the direction of gravity or a ground surface, rather than extending parallel with a ground surface from a connection position with an endoscope, as depicted in FIG. 17B.

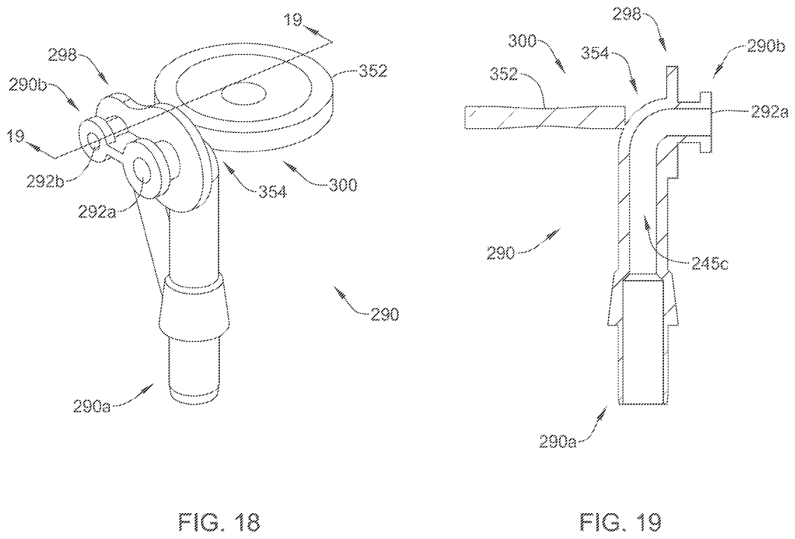

The connector 290 depicted in FIGS. 18 and 19 may include a stabilizing member 300 having a tab 352 and an elbow bend 354 between a first end 290a and a second end 290b of the connector 290 to facilitate tubing coupled to connector 290 being positioned tangential to an umbilical when the connector 390 is coupled to one or more fluid ports of the umbilical. In some cases, the tab may be engaged fingers of a user to facilitate connecting the connector 290 to one or more fluid ports and or to facilitate removing the connector 290 from one or more fluid ports and thus, may improve the stability the connector 290 over time as the connector 290 repeatedly engages and disengages the fluid ports.

FIG. 19 depicts a schematic cross-section view of the connector 290 taken along 19-19 in FIG. 18. As shown in FIG. 19, the elbow bend 354 may be ninety degrees or about ninety degrees, however, it is contemplated the elbow bend 354 may take on one or more other suitable angles. Further, the lumen or tubing 245c and/or other lumens of the connector 290 may follow the elbow bend 354 between the first end 290a and the second end 290b of the connector 290. Similar to as discussed above with respect to FIGS. 17A and 17B, the elbow bend 354 configuration of the connector 290 may reduce stress on the connector 290 and the tubing connected to the connector 290 relative to a configuration of the connector 290 depicted in FIG. 3 due to the first end 290a of the connector and tubing connected thereto being positioned in the direction of gravity or a ground surface, rather than extending parallel with a ground surface from a connection position with an endoscope, which may be the result when using a configuration of the connector 290 depicted in FIG. 3.

Further, as depicted in FIG. 19, the tab 352 may be substantially in-line with the ports 292 of the connector 290 and transverse or substantially transverse to the lumen or tubing 245c at the first end 290a of the connector 290. The positioning of the tab at a location in-line with or nearly in-line with the ports 292 of the connector 290 may facilitate using the tab 352 to apply a required force to the connector 290 and ports 292 to facilitate connection with fluid ports of an endoscope and/or for using the tab 352 to apply a required force to the connector 290 to facilitate removing the connector 290 from fluid ports of the endoscope.

Further, although not depicted in the Figures, the various configurations of the connector 290 may utilize one or more coupling components configured to couple the connector 290 to one or more of the features of the endoscope system 200 while the connector 290 is disengaged from the fluid ports of the endoscope 100. In some cases, the coupling feature may be coupled to tubing to maintain the tubing in a desired position while packaged.

The coupling component may take on any suitable configuration configured to couple to tubing and/or components of an endoscope system. Example configurations of the coupling component include, but are not limited to, a clip, a hook, a button, a hook and loop fastener, a magnetic feature on or embedded in the connector 290, and/or one or more other suitable configurations. In some cases, the coupling component may be particularly configured to mate with one or more other components of the endoscope system 200, but this is not required.

In operation, a user may disconnect the connector 290 from fluid ports of an umbilical. Once the connector 290 is disconnected, the coupling component may be coupled to a processor and/or other suitable feature of the endoscope system 200. Such a coupling component may reduce work for users as the user will not be required to hold the connector 290 between procedures and may prevent or mitigate contamination issues as the connector 290 may be secured to a known location between procedures.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A connector for connecting a gas/lens wash tubing to fluid lines of an endoscope, the connector comprising:
a core configured to couple to the gas/lens wash tubing;
a device coupling member coupled to the core, the device coupling member defining a first port configured to couple with a first fluid port of an endoscope and a second port configured to couple with a second fluid port of an endoscope; and
a stabilizing member configured to facilitate securing the device coupling member to the endoscope.

2. The connector of claim 1, wherein the stabilizing member comprises a ring in the first port.

3. The connector of claim 2, wherein the ring has an inner surface and the inner surface tapers toward a central axis of the first port.

4. The connector of claim 2, wherein the stabilizing member includes a ring and extends distally from the core into the first port.

5. The connector of claim 2, wherein the core and the stabilizing member are a unitary component and the device coupling member extends over a portion of the unitary component.

6. The connector of claim 1, wherein the stabilizing member comprises a strap and a strap connector, and the strap is configured to extend around an umbilicus of the endoscope and couple to the strap connector.

7. The connector of claim 1, wherein the stabilizing member comprises a slot extending between an outer surface of the device coupling member and the second port, and the slot facilitates engaging the second port with the second fluid port via rotation of the device coupling member.

8. The connector of claim 1, wherein the stabilizing member comprises:
an actuator; and
a retention component in communication with the actuator; and
wherein the retention component is in a first configuration relative to the first port when the actuator is in a first position and the retention component is in a second configuration relative to the first port when the actuator is in a second position.

9. The connector of claim 1, wherein the stabilizing member comprises:
a rigid outer member; and
engaging members extending from the device coupling member; and
wherein the first port is configured to extend around the first fluid port of the endoscope and the rigid outer member is configure to engage the locking members to secure the device coupling member to the endoscope.

10. A connector for connecting tubing to fluid ports of a medical device, the connector comprising:
a first end configured to couple with the tubing and including a first lumen and a second lumen, the second lumen is coaxial with the first lumen;
a second end including:
a first port in fluid communication with the first lumen and configured to engage a first fluid port of the medical device; and
a second port in fluid communication with the second lumen and configured to engage a second fluid port of the medical device; and
a stabilizing component configured to facilitate securing the second end to the medical device when the first port engages the first fluid port.

11. The connector of claim 10, wherein the stabilizing component is configured as a ring in the first port and the ring is configured to center the first fluid port within the first port and the second fluid port in the second port.

12. The connector of claim 11, wherein the ring is formed from a rigid material and the first port and the second port are at least partially formed from a resilient material.

13. The connector of claim 10, wherein the first lumen and the second fluid lumen form an elbow between the first end and the second end.

14. The connector of claim 10, wherein the stabilizing component comprises a tab extending transverse to an axis of the first lumen at the first end.

15. The connector of claim 10, wherein the stabilizing component comprises an elbow joint between the first end and the second end.

16. The connector of claim 13, wherein the first end is formed from a rigid material, the second end is formed from a resilient material, and the resilient material overlaps the rigid material.

17. The connector of claim 13, wherein the stabilizing component comprises a retention component configured to engage a component of an endoscope system.

18. A connector for connecting tubing to fluid ports of a medical device, the connector comprising:
a first end configured to couple with the tubing and including a first lumen and a second lumen, the second lumen is coaxial with the first lumen;
a second end including:
a first port in fluid communication with the first lumen and configured to engage a first fluid port of the medical device; and
a second port in fluid communication with the second lumen and configured to engage a second fluid port of the medical device; and
an actuator;
a retention component in communication with the actuator; and
wherein the retention component is configured to adjust to a fluid port engaging position in the first port in response to a first actuation of the actuator and adjust to a fluid port disengaged position in response to a second actuation of the actuator.

19. The connector of claim 18, wherein the retention component is biased to the fluid port engaging position.

20. The connector of claim 18, wherein a material forming the second end biases the retention component to the fluid port engaging position.
